(19) United States
(12) Patent Application Publication
(10) Pub. No.: US 2009/0246278 A1
(43) Pub. Date: Oct. 1, 2009

(54) USE OF ESSENTIAL OILS TO REDUCE BENZODIAZEPINE REQUIREMENTS DURING MEDICAL PROCEDURES

(76) Inventor: Kamyar Malek HEDAYAT, San Diego, CA (US)

Correspondence Address: AMIN Talati, LLC, 444 NORTH ORLEANS STREET, SUITE 400, CHICAGO, IL, 60654, US

(21) Appl. No.: 12/415,579

(22) Filed: Mar. 31, 2009

Related U.S. Application Data
(60) Provisional application No. 61/040,822, filed on Mar. 31, 2008.

(51) Int. Cl. A61K 9/10; A61K 36/28

(52) U.S. Cl. 424/484; 424/764

(57) ABSTRACT

A method to reduce or eliminate the need for medicaments such as benzodiazepine and narcotics in noxious medical procedures, such as intravenous needle insertion, mechanical ventilation and intubation procedures, includes topically administering to the patient an effective amount of an essential oil composition including at least one essential oil selected from Roman Chamomile (Anthemis nobilis) essential oil, Sandalwood (Santalum album) essential oil and Galbanum (Ferrula gummosa) essential oil, and/or their components or other essential oils with similar effects or properties.

This application claims the benefit of earlier filed Provisional U.S. Patent Application Ser. No. 61/040,822 filed on Mar. 31, 2008.

### FIELD OF THE INVENTION

The present invention generally relates to a method and formulation for topical and internal application of essential oils in order to reduce requirements of medicaments during medical procedures. More particularly, the present invention relates to a method and formulation for topical and internal application of essential oils administered to reduce the requirements for narcotics, benzodiazepine and other drugs during noxious medical procedures such as intravenous needle insertion, intubation and other procedures or events which increase anxiety and discomfort in a person, particularly in populations where dependence and withdrawal symptoms are likely to occur.

### BACKGROUND OF THE INVENTION

Critically ill patients often undergo noxious interventions such as intravenous needle insertion, mechanical ventilation and other procedures or events which increase anxiety and discomfort in a person. To tolerate these unpleasant stimuli patients, infants and children in particular, are sedated with continuous infusions of benzodiazepines and narcotics.¹ Both insufficient and excessive use of sedatives can be harmful to patients, causing increased mortality and prolonged mechanical ventilation.²

Continuous sedation with benzodiazepine or benzodiazepine derivatives, such as midazolam, is commonly employed in hospitals, for example in the pediatric intensive care unit. While its use is essential to achieve compliance with painful life-saving therapies, benzodiazepines and other narcotics, can have multiple adverse effects.

Up to one third of pediatric patients may experience withdrawal symptoms,³ with abrupt discontinuation resulting in tremors, agitation, hallucinations, and seizure.⁴ Reports of prolonged sequelae in infants, such as dystonia and visual problems, have been reported.⁵ To avoid these complications, many children require maintenance therapy with long-acting benzodiazepines, delaying discharge home. Benzodiazepines also interfere with rapid eye movement sleep patterns, resulting in a profound sense of fatigue, and may lead to disorientation or agitation after days remaining in a drug-induced state of sleep. Prolonged sedation leads to postextubation paresis requiring physical therapy. Prolonged periods of stasis lead to lymph edema and third spacing, requiring diuretics, which lead to electrolyte derangements.

The ability to reduce the amount of benzodiazepines used during critical illness may help reduce the incidence or severity of these side effects and sequelae.

Thus, in view of the above, there is a need and a demand for a method and/or formulation for alleviating the reliance on benzodiazepine and narcotics in patients, particularly children, who require sedation for medical treatments, namely intravenous needle insertion, mechanical ventilation and intubation or any other procedures or events which increase anxiety and discomfort in a person. There is a further need and a demand for a method and/or formulation which is inexpensive to produce and easy to administer.

### SUMMARY OF THE INVENTION

A general object of the invention is to provide a composition which can be used as an adjuvant to benzodiazepines and/or narcotics to achieve a sufficient or desired level of sedation in a patient.

A more specific object of the invention is to overcome one or more of the problems described above.

The general object of the invention can be obtained, at least in part, via a method for topically or internally applying essential oils in order to reduce or eliminate the need for medicaments such as benzodiazepine and narcotics in medical procedures, particularly mechanical ventilation and intubation procedures, intravenous needle insertion or other procedures or events which increase anxiety and discomfort in a person. The method includes topically or internally administering to the patient an effective amount of an essential oil composition including at least one essential oil selected from Roman Chamomile (Anthemis nobilis) essential oil, Sandalwood (Santalum album) essential oil and Galbanum (Ferrula gummosa) essential oil. The essential oil(s) can be used alone or combination with one or more essential oil components selected from alpha-pinene, beta-pinene, camphene, sabinene, myrcenen, 1,8-cineole, y-terpinene, caryophylle, propylangelate, butyl angelate, santalol, satyl acetate, santalene, monoterpines, pyrazines, galbanolene, and galbanum pyrazine. At least partial reliance on medicaments such as benzodiazepine or narcotics during medical procedures is alleviated via topical or internal application of the essential oil composition before, during and/or after medicaments would normally be administered for the procedure.

The invention further comprehends multiple dosing regimens based on the duration of the medical procedure, age of the patient and severity of illness.

The invention also comprehends topical application of an essential oil composition that is applied via massage or other means directly to the patient.

The invention additionally comprehends passive inhalation of a liquid essential oil composition that is applied to areas around the patient, such as skin, clothing or bedding.

Moreover, the invention comprehends consumption of the essential oil composition.

As used herein, the term “essential oil” refers to any concentrated, hydrophobic liquid containing volatile hydrocarbon compounds derived from plants such as aromatic herbs, aromatic plants, or single compounds isolated there from.

As used herein, the terms “benzodiazepine” and “benzodiazepines” refer to benzodiazepine drugs and their derivatives including, but not limited to, midazolam.

As used herein the term “drop” refers to a liquid volume equivalent to about 50 microliters (50 μl) to about 80 μl. For example, three (3) drops of a formulation in accordance with the invention has an equivalent liquid volume of between about 150 μl and 240 μl.

Other objects and advantages will be apparent to those skilled in the art from the following detailed description taken in conjunction with the drawings and examples.

### BRIEF DESCRIPTION OF THE FIGURE

FIG. 1 is a graphical representation of alterations in bispectral index scores and physiological parameters pre- and post-essential oil application in a patient under benzodiazepine mediated sedation.

### DETAILED DESCRIPTION

Benzodiazepines and narcotics are a mainstay of continuous sedation in the intensive care unit. Benzodiazepines such as midazolam bind to postsynaptic γ-aminobutyric acid (GABA) receptors allowing for the inhibitory neurotransmitter GABA to bind to their receptors. This results in various effects such as hypnosis, sedation and muscle relaxation depending on the location of the receptor and its subunits (α, β, and γ). All of the current benzodiazepines are known to affect GABA_{A} receptors.⁶

Many essential oils have been found to interact with gamma-aminobutyric acid (GABA) receptors, resulting in subjectively and objectively measured feelings of calmness, relaxation, and sedation with improved sleep in animal and human trials.

A number of studies have demonstrated a reduction in dynamic physiologic variables during topical application of essential oils in experimental conditions in healthy adults. However, such studies are not accompanied by objective evidence of increased sedation.⁷ Additionally, a study comparing unscented massage to massage with essential oils in adult intensive care patients showed reduced subjective evaluation of anxiety in the aromatherapy group but no improvement in physiologic variables in either group.⁸

However, it has been surprisingly discovered that essential oils, when used as an adjuvant to benzodiazepine therapy, can result in increased sedation levels at reduced benzodiazepine requirements. For example, a topically applied essential oil formulation was found to objectively and effectively increase sedation levels in a patient undergoing benzodiazepine therapy as evidenced by reduction in bispectral index scores as well as in several associated physiological parameters such as heart rate, systolic blood pressure, and diastolic blood pressure.

Essential oils are volatile plant oils which are polychemical in nature, although 2 to 4 compounds will make up 60% to 90% of an oil's constituents.⁹ Essential oils are hydrocarbon structures with functional groups such as esthers, phenols, ketones, alcohols and oxides similar to synthetic pharmaceuticals.¹⁰

Three essential oils have traditionally been used for their purported relaxing effects.¹¹

Roman chamomile essential oil decreases sleep latency via a GABA-dependent mechanism without affecting REM state sleep¹² and stimulates alpha waves on EEG which is associated with a state of relaxation.¹³

Topical sandalwood essential oil use in a clinical trial reduced sympathetic response while creating a subjective feeling of calm awareness.¹⁴

α- and β-pinene, found in Galbanum essential oil, have been found to exert GABA-ergic properties.¹⁵ These effects are antagonized by the benzodiazepine antagonist flumazenil, suggesting the interaction with GABA receptors is primarily responsible for the effects seen.⁶

Many other essential oils have been found to interact with gamma-aminobutyric acid (GABA) receptors, resulting in subjectively and objectively measured feelings of calmness, relaxation, and sedation with improved sleep in animal and human trials. Thus, essential oils may serve a complementary role in the intensive care unit in augmenting sedation while reducing benzodiazepine and narcotic requirements.

Essential oils such as those described herein offer a novel and inexpensive treatment option for patients who prefer a decrease in the need for benzodiazepines and narcotics or those with an increased likelihood of dependence or where withdrawal symptoms are likely to occur.

In accordance with one embodiment, an essential oil formulation can include or consist of one or more essential oils derived from Roman Chamomile, Sandalwood and Galbanum. Suitably, the essential oil formulation can include each oil in a 1:1 ratio. For example, an essential oil formulation can include Roman Chamomile and Sandalwood in a 1:1 ratio.

In accordance with another embodiment, an essential oil formulation can include or consist of a combination of Roman Chamomile, Sandalwood and Galbanum at a concentration of 7.5% in a base of jojoba oil.

In an alternative embodiment, an essential oil formulation can include one or more of the essential oils of Roman Chamomile, Sandalwood and Galbanum alone or in combination with one or more of the compounds alpha-pinene, beta-pinene, camphene, sabinene, myrcenen, 1,8-cineole, y-terpinene, caryophylle, propylangelate, butyl angelate, santalol, satyl acetate, santalene, monoterpines, pyrazines, galbanolene, and galbanum pyrazine.

In another embodiment, the essential oil formulation can include or consist of essential oils derived from Roman Chamomile, Sandalwood and Galbanum, alone or in combination with other essential oils that may lead to similar effects. Such other essential oils include, but are not limited to the essential oils neroli (Citrus aurantium), linalool, agarwood, bergamot, cedarwood, geranium, lemon, rosemary, clary sage (Salvia sclarea), helichrysum (H. angustfolium), sweet marjoram (Origanum majorana) and/or vetiver (Vetiveria zizanioides).

In accordance with certain embodiments, the essential oil formulation can further include a carrier matrix. Suitably, the carrier matrix is non-aromatic and can be in the form of a solid, liquid, gel, cream, or other emulsion. For example, the carrier matrix can include or consist of a non-aromatic liquid selected from water, pharmaceutically acceptable solvents (e.g., ethanol), unscented oils, lightly scented oils, and dispersions or emulsions thereof.

In accordance with one embodiment, the carrier matrix can include or consist of a non-aromatic carrier oil. For example, the essential oil formulation can include an unscented massage oil or other unscented or lightly scented, cosmetically acceptable carrier oil including, but not limited to, vegetable oils, grapeseed oil, avocado oil, jojoba oil, wheat germ oil, sunflower oil, or combinations thereof. For topical applications, the carrier matrix should be suitable for use on a patient's skin particularly the skin of the chest, back, and neck. For internal use, the carrier matrix should be suitable for human consumption.

An essential oil composition suitable for application to a surface of a patient's skin, clothing and/or bedding (i.e., a topical composition) can include or consist of about 0.5% to about 10% by volume of the essential oil composition. In accordance with certain embodiments, a topical essential oil composition used to reduce or eliminate the need for medicaments such as benzodiazepine and narcotics in medical procedures, particularly intravenous needle insertion, mechanical ventilation and intubation procedures can include about 1% to about 8% by volume of an essential oil composition including or consisting of: about 25% to about 40% by volume Roman Chamomile; about 25% to about 40% by volume Sandalwood; and about 25% to about 40% by volume Galbanum. The essential oil(s) can be mixed with or dispersed in a carrier matrix such as, for example, a non-aromatic carrier oil (i.e., an unscented or lightly scented oil).

An essential oil composition suitable for internal application (i.e., an internal composition) can include or consist of about 0.5% to about 10% by volume of the essential oil composition. In accordance with certain embodiments, an internal essential oil composition used to reduce or eliminate the need for medicaments such as benzodiazepine and narcotics in medical procedures, particularly intravenous needle insertion, mechanical ventilation and intubation procedures can include about 1% to about 8% by volume of an essential oil composition including or consisting of: about 25% to about 40% by volume Roman Chamomile; about 25% to about 40% by volume Sandalwood; and about 25% to about 40% by volume Galbanum. The essential oil(s) can be mixed with or dispersed in a carrier matrix suitable for oral consumption or other internal consumptions, for example passive and active inhalation.

A method to reduce or eliminate the need for medicaments such as benzodiazepine and narcotics in medical procedures which increase anxiety and discomfort in a person, particularly mechanical ventilation, intravenous needle insertion, and intubation procedures includes administering to a patient having such requirements an essential oil composition including or consisting of an essential oil and/or an essential oil component. The essential oil can be selected from Roman Chamomile, Sandalwood, Galbanum, or combinations thereof. The essential oil component can be selected from the group consisting of alpha-pinene, beta-pinene, camphene, sabinene, myrcenen, 1,8-cineole, y-terpinene, caryophylle, propylangelate, butyl angelate, santalol, satyl acetate, santalene, monoterpines, pyrazines, galbanolene, and galbanum pyrazine, and combinations thereof.

In accordance with certain embodiments, about 25 drops (about 1.25 ml) of the essential oil composition can be mixed with or dispersed in about 2 ml of a carrier oil such as a non-aromatic massage oil suitable for cosmetic purposes. The essential oil composition may be internally consumed or applied to a surface of a patient's skin, clothing and/or bedding as needed before, during and/or after a need for benzodiazepines and narcotics arises.

In accordance with a further embodiment, a method for reducing a patient's benzodiazepine requirement during, for example, intubation includes administering an effective amount of an essential oil composition including an oil blend dispersed in a carrier matrix. The oil blend includes about 25% to about 40% of Roman Chamomile, about 25% to about 40% Sandalwood, and about 25% to about 40% Galbanum. The patient's level of sedation is increased and the patient's benzodiazepine requirement is decreased. The increase in the patient's level of sedation can be objectively measured according to a decrease in a bispectral index score (BIS), a decrease in heart rate, a decrease in systolic blood pressure, and/or a decrease in diastolic blood pressure.

### EXAMPLE

Example Formulation: A 2.5 ml topical blend is prepared as follows:

TABLE 1

| Essential Oil | ml | % by Volume |
|---|---|---|
| Roman Chamomile | 0.0625 | 2.5 |
| Sandalwood | 0.0625 | 2.5 |
| Galbanum | 0.0625 | 2.5 |
| Jojoba Oil | 2.3125 | 92.5 |
| Total | 2.5 | 100.00 |

The essential oil formulation can be administered such as via topical application, internal application or massage in a carrier of jojoba oil.

### EXPERIMENTAL RESULTS

An 18-month-old previously healthy female (“AP”) was admitted with respiratory distress secondary to adenovirus pneumonia. Her pneumonia progressed to respiratory failure, requiring endotracheal intubation and mechanical ventilation within 48 hours of admission. She failed extubation on day eight of mechanical ventilation secondary to subglottic edema caused by thrashing from inadequate sedation. After reintubation, attempts to resume sedation at lower levels were not tolerated by the patient. Midazolam infusion resumed at 0.3 mg/kg/hr. In place of fentanyl 5 mcg/kg/hr, a morphine infusion was started at 0.1 mg/kg/hr and quickly titrated to 0.2 mg/kg/hr. AP continued to be agitated and a paralytic infusion of vecuronium was initiated to prevent further subglottic irritation from thrashing.

A bispectral index (BIS) monitor was placed to monitor AP's level of sedation while receiving a continuous paralytic infusion. The BIS monitor is a processed continuous electroencephalogram which objectively measures depth of sedation and lack of recall.¹⁷ Bispectral index monitoring is used intraoperatively and in the intensive care unit when a patient is paralyzed and subjective measures of discomfort cannot be relied on to gauge discomfort and subjective measures of discomfort cannot be relied on to gauge discomfort. A goal BIS score of 40 to 60 was chosen to achieve minimal recall and adequate sedation. After 45 hours of sedation and paralysis on advancing doses of midazolam and morphine (at the doses noted above), the mean BIS score was 59 (40-77). The mother asked permission to massage her child while receiving mechanical ventilation.

The massage oil consisted of a 7.5% total concentration of Roman chamomile (Anthemis nobilis), Sandalwood (Santalum album), and Galbanum (Ferrula gummosa) in a base of jojoba oil (Simondia chinensis). The mother was instructed to apply 2.5 ml of the essential oil blend every six to eight hours to her daughter's arms, legs, and chest. AP received two applications nine hours apart. The second application was given when AP appeared agitated after a sponge bath.

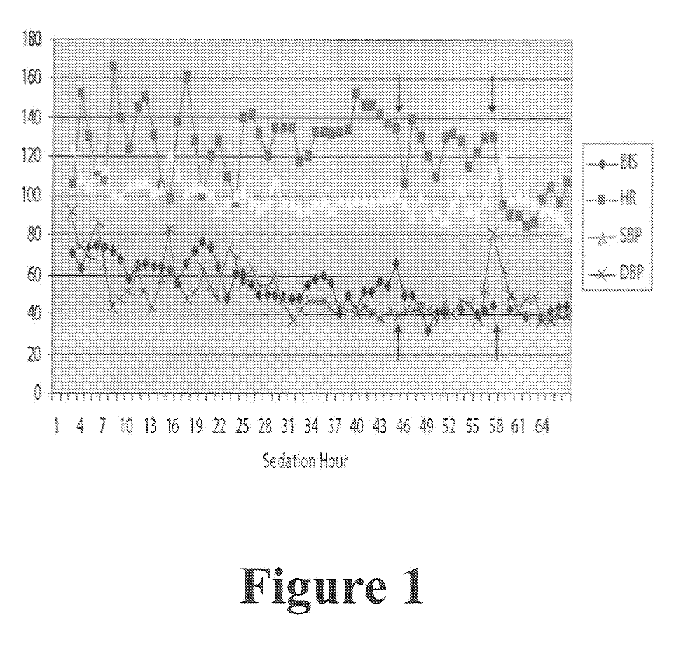

The BIS declined from 50 to 44 within one hour of the first application on the legs, reaching at nadir at two hours postapplication of 32. The midazolam infusion was reduced by 50% to 0.1 mg/kg/hour within two hours of the first application and remained at that level for the remainder of her mechanical ventilation (cf, FIG. 1). Mean BIS score postapplication was 41 for the remaining 16 hours before extubation despite the 50% reduction in midazolam infusion. No changes were made to the vecuronium or morphine infusions (cf, FIG. 1).

Mean heart rate was 130 beats per minute (bpm) (96-166) for the proceeding 45 hours preapplication, mean 110 bpm (85-132) postapplication. Mean systolic blood pressure (SBP) was reduced from 101 mmHg (93-124) to 96 mmHg (83-104), mean diastolic blood pressure (DPB) was reduced from 53 mmHg (39-92) to 47 mmHg (38-83) preapplication and postapplication, respectively. (cf, FIG. 1).

AP required a six day weaning course of lorazapam and methadone for signs of withdrawal. She showed signs of severe fatigue for the first 72 hours postextubation. She also showed signs of global paresis requiring physical therapy until the time of her discharge and she was discharged home 18 days after admission, six days after being successfully extubated.

Children in the intensive care unit often require prolonged periods of sedation. However only a subset requires paralysis as well.¹⁸ A number of clinical sedation scores have been developed based on a combination of physiologic variables and behavioral responses. Such scores cannot be used to adequately gauge depth of sedation in paralyzed patients.¹⁹ Bispectral index monitoring has been found to be accurate in predicting degree of sedation in adult patients receiving midazolam²⁰ as well as in critically ill children.^{21,22} The BIS monitor represents an objective measurement of depth of sedation in critically ill patients.

One study found a BIS score of 50 to 70 to indicate moderate sedation in children.¹⁸ However, our patient's BIS averaged 59 prior to application of the essential oils and she continued to appear agitated based on physiologic parameters. Within one hour of topical application of essential oils, AP's BIS score was reduced to 41 allowing her benzodiazepine infusion to be decreased by 50%. A score of 41 correlates with deep sedation in pediatric patients.¹⁸ In addition to improved sedation by BIS score, our patient had favorable physiologic responses to the essential oil application. An 18% reduction in average heart rate was noted with modest improvements in SPB and DPB and no adverse changes in oxygenation.

I claim:

1. A method to reduce or eliminate the need for medicaments such as benzodiazepine and narcotics in medical procedures, comprising:
administering to the patient an effective amount of an essential oil composition including:
at least one essential oil selected from the group consisting of Roman Chamomile, Sandalwood, and Galbanum; or
at least one oil component selected from the group consisting of alpha-pinene, beta-pinene, camphene, sabinene, myrcenen, 1,8-cineole, y-terpinene, caryophylle, propylangelate, butyl angelate, santalol, satyl acetate, santalene, monoterpines, pyrazines, galbanolene, and galbanum pyrazine, and combinations thereof; or
combinations thereof.
wherein the patient's need for medicaments to maintain sedation is at least partially reduced by the administration of the essential oil composition.

2. The method according to claim 1, wherein the essential oil composition comprises at least two essential oils, each oil present in a 1:1 ratio.

3. The method according to claim 1, wherein the effective amount of the essential oil composition is administered in a quantity comprising about 62.5 microliters (μl) to about 187.5 μl of at least one essential oil.

4. The method according to claim 1, wherein the administering step comprises topically applying the essential oil composition.

5. The method according to claim 4, wherein in the essential oil composition is administered to the patient via massage.

6. The method according to claim 4, wherein the essential oil composition is topically applied to areas around the patient, the areas comprising the patient's skin, clothing, bedding, other areas near the patient, and combinations thereof.

7. The method according to claim 1, wherein the essential oil composition is internally applied to the patient through oral consumption or inhalation.

8. The method according to claim 1, wherein the essential oil composition further comprises a carrier matrix.

9. The method according to claim 8, wherein the essential oil composition comprises about 25 μl to about 250 μl of at least one essential oil, essential oil component, or combination thereof, dispersed in about 1 ml to about 5 ml of the carrier matrix.

10. A method to reduce a patient's need for medicaments such as benzodiazepine and narcotics during medical procedures, comprising:
administering to the patient an effective amount of an essential oil composition including:
at least one essential oil selected from the group consisting of Roman Chamomile, Sandalwood, Galbanum, neroli (Citrus aurantium), linalool, agarwood, bergamot, cedarwood, geranium, lemon, rosemary, clary sage (Salvia sclarea), helichrysum (H. angustfolium), sweet marjoram (Origanum majorana) and/or vetiver (Vetiveria zizanioides); and combinations thereof, and
optionally, a carrier matrix;
wherein the patient's need for medicaments to maintain sedation is at least partially reduced by topical or internal application of the essential oil composition.

11. The method according to claim 10, wherein the essential oil composition comprises about 0.5% to about 10% by volume of at least one essential oil.

12. The method according to claim 10, wherein the essential oil composition comprises a blend of Chamomile, Sandalwood, and Galbanum oils in a 1:1:1 ratio.

13. The method according to claim 10, wherein the carrier matrix comprises a non-aromatic liquid selected from the group consisting of water, pharmaceutically acceptable solvents, unscented oils, lightly scented oils, and dispersions or emulsions thereof.

14. The method according to claim 10, wherein the essential oil composition comprises at least about 90% by volume carrier matrix.

15. A method to reduce a patient's benzodiazepine requirement during intubation, comprising:
administering to the patient an effective amount of an essential oil composition including:
an oil blend dispersed in a carrier matrix, the oil blend including
about 25% to about 40% by Roman Chamomile;
about 25% to about 40% Sandalwood; and
about 25% to about 40% Galbanum;
wherein as the patient's level of sedation is increased and the patient's benzodiazepine requirement is decreased.

16. The method according to claim 15 wherein the effective amount of the essential oil composition comprises about 25 μl to about 250 μl of the oil blend dispersed in about 1 ml to about 5 ml of the carrier matrix.

17. The method according to claim 15, wherein the essential oil composition is topically administered in one or more doses.

18. The method according to claim 17, wherein a dose comprises between 250 μl and 2.5 ml of the essential oil composition.

19. The method according to claim 17, wherein the one or more doses are administered at a single time up to, and including, continuously during the need for sedation.

20. The method according to claim 15, wherein an increase in a patient's level of sedation is objectively determined using one or more of a decrease in a bispectral index score, heart rate, systolic blood pressure, and diastolic blood pressure.
